(19) United States
(12) Patent Application Publication
(10) Pub. No.: US 2012/0040581 A1
(43) Pub. Date: Feb. 16, 2012

(54) TEMPLATE-SUPPORTED METHOD OF FORMING PATTERNS OF NANOFIBERS IN THE ELECTROSPINNING PROCESS AND USES OF SAID NANOFIBERS

(75) Inventor: Gyeong-Man Kim, Donostia - San Sebastian (ES)

(73) Assignee: CENTRO DE ESTUDIOS INVESTIGACIONES TECNICAS DE GIPUZKOA, San Sebastian (ES)

(21) Appl. No.: 13/262,577

(22) PCT Filed: Mar. 31, 2010
(86) PCT No.: PCT/EP10/54350

§ 371 Date: Sep. 30, 2011

(30) Foreign Application Priority Data
Apr. 1, 2009 (DE) 10 2009 015 226.1

(51) Int. Cl. D04H 1/74; B29C 47/08; D02G 3/36; C08G 63/00; C08G 69/02; C08G 65/00; C08F 10/00; C08G 64/00; C08G 18/00; C08B 37/00; C08G 63/08; C08F 112/08; C08F 120/10; C08F 120/44; C08G 59/00; C08G 73/10; C12N 9/96; C08G 63/91; C08G 69/48; C08G 18/30; C08G 59/14; B29C 67/20; B82Y 30/00

(52) U.S. Cl. 442/330; 264/413; 442/370; 428/375; 425/174.8 E; 528/271; 528/310; 528/425; 526/348; 528/370; 528/85; 536/123.1; 528/354; 526/346; 526/319; 526/341; 528/403; 525/54.1; 525/54.2; 435/188; 525/418; 525/420; 525/55; 525/461; 525/453; 525/523; 977/700

(57) ABSTRACT

The invention relates to a method for producing two- and three-dimensionally structured, microporous and nanoporous webs made up of nanofibers in any form with a very high covering or depositing degree of the fibers by means of a predefined conductive mold (template) as a collector and to the use of the webs according to the invention. The three-dimensional structure formation can be influenced in a directed manner by the deposition density of the nanofibers generated by means of an electrospinning process, which deposition density is adjustable through the accumulation time of the fibers.

The invention relates to a method for producing two- and three-dimensionally structured, microporous and nanoporous webs made up of nanofibers in any form with a very high covering or depositing degree of the fibers by means of a predefined conductive mold (template) as a collector and to the use of the webs according to the invention. The three-dimensional structure formation can be influenced in a directed manner by the deposition density of the nanofibers generated by means of an electrospinning process, which deposition density is adjustable through the accumulation time of the fibers.

Modern, synthetically-produced polymer fibers have diverse, innovative applications, such as, for example, for multifunctional textile materials with greater breathing activity and weather resistance, as separating or storage means for gases, liquid or suspensions of particles in processing and safety technology, as optical conductors for telecommunication, as reinforcement components in super-lightweight composites, in the public health sector and in the field of sports and leisure.

There are already a number of synthesis pathways and production methods for generating one-dimensional structures made up of different polymers in fibers, wires, rods, bands, spirals, rings and others. The polymer fibers often used for that purpose are traditionally produced by dry or wet spinning processes in molten mass, the typical fiber diameters being in the order of magnitude of from approximately 5 μm to 500 μm. The diameter of these fibers generated by means of conventional processing techniques is however downwardly limited due to reasons of the processing technique.

However, in recent years there has been an essential contribution to the technological advance in producing ultrathin fibers based on nanotechnology. It is also necessary to include the electrospinning process, which represents a simple, rapid and economical method for producing nanofibers, particularly thin polymer fibers, with a diameter of up to a few nanometers, taking place, in contrast with conventional mechanical processes, the contact-free drawing of the fibers by applying an external electric field.

In the electrospinning process, an electric field is applied between a fine capillary nozzle, for example the syringe cannula, and a collector electrode, such as, for example, a conductive plate, for counteracting and finally overcoming the surface tension of the drop of a polymer molten mass or solution coming out of the capillary nozzle. In the event that the viscosity of the polymer molten mass or solution is within a specific optimal range, the drop coming out of the capillary nozzle deforms and when it reaches a critical electric potential it is drawn to yield a fine filament, the so-called jet (FIG. 1).

This electrically-charged jet, now continuously extracting new polymer molten mass or solution from the capillary nozzle, is then accelerated in the electric field towards the counter electrode. In this regard, it is subjected in a very complex manner to bending instability (the so-called whipping mode), turned with force and highly drawn.

The jet solidifies during its flight towards the counter electrode by means of the evaporation of the solvent or by means of cooling, such that in the period of a few seconds continuous fibers are generated linked with one another with typical diameters of a few nanometers to several micrometers. These fibers accumulate on the counter electrode in the form of a web, the nonwoven mat, and are additionally processed (document U.S. Pat. No. 197,550; Kenawy et al., Biomaterials 24:907 (2003); Deitzel et al., Polymer, 42:8163 (2001); Reneker et al., Nanotechnology 7:216 (2000)).

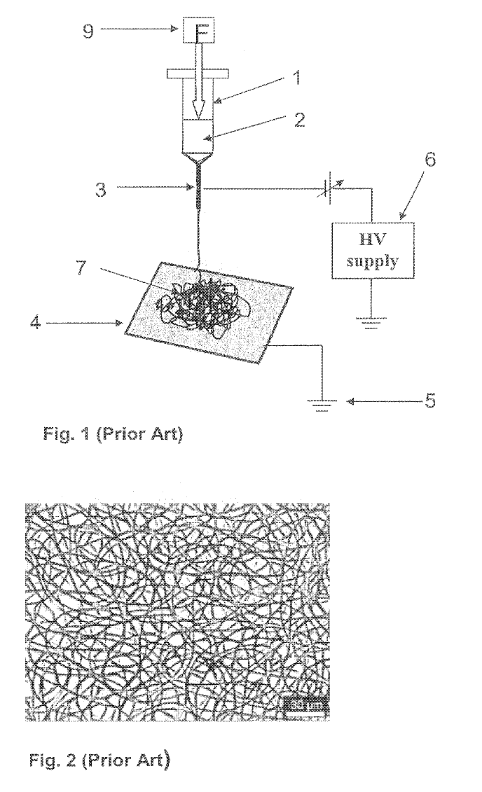

Generally, the jet extracted from the capillary nozzle exerts a strong interaction between the electric charges in the jet and the external electric field, whereby the path of the jet cannot be clearly defined. If a continuous plate made of a conductive material is used as a collector electrode, a web of nanofibers arranged on top of one another or next to one another without any orientation on the collector electrode is obtained (FIG. 2).

Due to its high length-thickness ratio and therefore its high specific surface area and its functionalization capacity by means of a surface treatment or nanoparticles, the polymer nanofibers produced in the electrospinning process have incredible possibilities for generating blends with completely novel “customized” properties that cannot be attained with conventional processes, such as, for example, for special textile materials, as nanostructured reinforcement elements, for membrane-based separators, for sensors, for the immobilization of biological messengers, for example DNA, RNA, enzymes and drugs, and in the fields of tissue engineering or regenerative medicine.

Two approaches are generally known for obtaining spun fibers with an order of magnitude. On one hand, one approach is to modify the collector, such as, for example, a rotating drum, wheel-shaped reels or metal frames. On the other hand, another approach is to manipulate the electric field, for example with the conductive electrodes located in parallel on a non-conductive collector electrode or with several electric lenses arranged parallel to one another, perpendicular to the collector electrode (document U.S. Pat. No. 4,689,186; R. Dersch et al., J. Polym. Sei. Part A: Pol. Chem., Vol. 41, 545-553).

However, the orientation of the fibers with the aforementioned processes is only possible one-dimensionally, two- and three-dimensional structures cannot be generated with them. However, there is a greater difficulty in these processes, specifically even though the fibers thus produced are oriented more or less parallel to one another the distances between the individual fibers can barely be controlled. The percentage of fibers with the same orientation is referred to as the degree of orientation and is indicated as a certain percentage. These processes known for orienting nanofibers further have a number of additional drawbacks, including a complicated construction of the spinning facilities and the need for several work steps and therefore a greater expenditure in terms of time and cost.

Document US 26308509 B1 discloses a device for generating textile fibers by electrospinning. In this regard, the nanofibers are spun to increase resistance with textile fibers to yield linear assemblies in the form of filaments referred to as yarns. These yarns can then be processed by means of textile treatment processes, such as weaving, braiding or knitting into two- or three-dimensional fabrics.

Furthermore, document WO 2008/049250 A1 discloses a method for producing microbicidal electrospun polymer fibers with polyethylenimine nanoparticles for textile applications. In this regard, the polymer fibers are spun with derived polyethylenimine nanoparticles and consequently an antibacterial or antifungal effect is achieved. The same effect is achieved by means of spinning polymer fibers with honey in encapsulated form, as disclosed in document WO 2008/049251 A1.

Document WO 2008/049397 A2 discloses a method for subjecting water-soluble polymers to electrospinning to yield a water-insoluble polymer fiber. In this regard, polyelectrolytes with opposite charges are spun in an aqueous solution by means of electrospinning to yield a water-insoluble polymer fiber.

Document DE 10 2007 040 762 A1 discloses a device and a method for producing electrically conductive nanostructures by means of electrospinning. In this regard, the electrically conductive particles are spun together with the spinning liquid to yield linear conductive structures. In one embodiment, the electrically conductive nanostructures can be generated by means of the subsequent treatment with conductive particles. It further discloses that the generated nanofiber is deposited on the collector with a directed orientation and high spatial precision. To that end, the spinning capillary and/or the substrate mount are mobile and their movement relative to one another is controlled by means of a computer. The structures generated with this method do not, however, have the necessary spatial precision, for example, for use in microsystems technology. Precision depends in this regard on the relative movement that can be made, on the precision of the operating unit and of the optical detection unit which supplies to the computer the necessary information necessary for the relative movement. The results that can thus be obtained furthermore are not reproducible precision-wise with respect to the spatial orientation of the deposited fibers. The disclosed method further requires an enormous expenditure in time and cost.

Document WO 2009/010443 A2 discloses a method for producing nanostructures and mesostructures by means of electrospinning colloidal dispersions containing at least one water-insoluble polymer. In this regard, the water-insoluble polymer is spun in an aqueous solution to yield a fiber, the glass transition temperature of the water-insoluble polymer being from a maximum of 15° C. above to a maximum of 15° C. below the operating temperature. The use of solvents can thus be greatly done away with. However, the webs and fibers produced with this method also present reduced precision with respect to deposition.

Due to the complicated interactions in the process parameters, for example viscosity, surface tension, conductivity, electric field intensity, aerodynamic drag and gravitation, the window of the electrospinning process is very limited. Furthermore, the fibers in the nonwoven mats have all the possible orientations, such that the use of these webs has been limited until now to special applications in which fibers with random orientation are also acceptable. A typical example of this is applications in the filter industry.

For valuable applications, for example both in microelectronics and photonics, and in culturing special tissues and organs, the defined generation of well-ordered one-, two- and three-dimensional structures, in which the fibers are highly oriented is indispensable.

The processes mentioned up to this point have the drawback that in order to orient the fibers, the forming matrix must be conserved. Therefore, it is not possible to obtain by means of the known processes a free web with respect to the manageability for the transfer thereof for additional work steps, to produce the final valuable products.

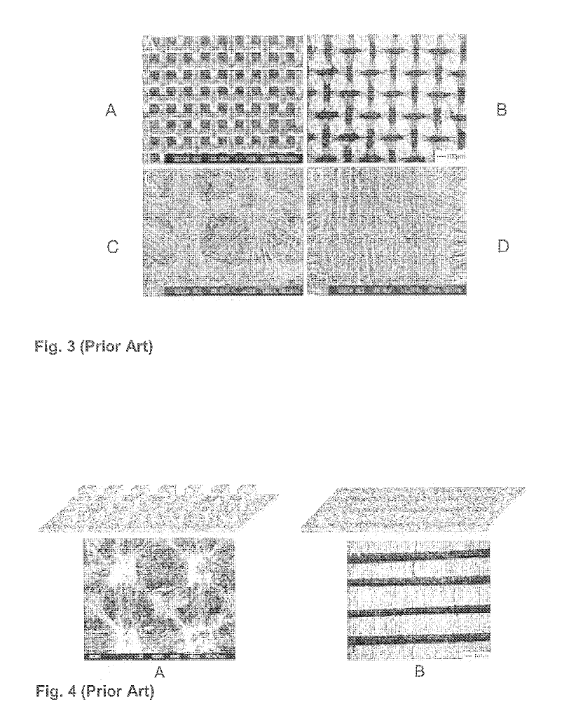

A method for generating patterns by means of electrospinning is further know, a predefined template being used (D. Zhang et al., Adv. Mater. 2007, 19, 3664-3667). This document discloses that the deposition of the nanofibers further shows a random arrangement. By simply using elevations in the predefined collector, better orientations can be obtained (FIG. 3), the degree of orientation depending on the separation of the elevations. In the case of too large of a separation, a chaotic deposition furthermore occurs (see FIG. 3C in particular). This effect is explained in that the coulometric interaction is inversely proportional to the separation between the capillary and the collector. Given that the coulometric interactions are an essential driving force of controlled deposition, therefore a deposition preferably occurs in the area between the elevations (FIG. 4). The method presented according to this works with the corresponding elevations in the collector to achieve a preferred orientation of the fibers.

As is evident from FIGS. 3 and 4 and from the preceding description, although an improved patterning is possible with the method thus disclosed, there is also a deposition of the jet in the intermediate space of the template, which counteracts the desired high covering or depositing degree of the nanofibers.

In addition, JP 2006 283241 A, JP 2007 303021 A, US 2005/104258 A1, the article Zhang, Darning, Chong, Jiang: “Patterning of Electrospun Fibres Using Electroconductive Templates”, Advanced Materials, Volume 19, Issue 21, November 2007, pages 3664-3667 and U.S. Pat. No. 3,280,229 A describe general methods for producing structured, microporous and nanoporous webs made up of nanofibers by means of electrospinning as well as general devices for performing such methods, from which the current invention emanates.

It is therefore highly desirable to develop a method whereby not only can the fibers be deposited in a controlled manner on a certain position to allow the specific structuring of the application of the fibers that will be spun, but the webs thus produced can also be additionally transferred to a substrate without damaging them.

The objective of the present invention therefore consists of indicating a method and a device which allow producing two- and three-dimensionally structured, microporous and nanoporous webs made up of nanofibers in any form with a very high covering or depositing degree of the fibers and consequently opening up new application possibilities of the microporous and nanoporous webs generated.

The objective is solved by means of the independent claims. Advantageous configurations are indicated in the dependent claims.

According to the invention, the production of two- and three-dimensionally structured, microporous and nanoporous webs made up of nanofibers in any form with a very high covering or depositing degree of the fibers takes place by means of electrospinning using a predefined conductive mold (template) as a collector, which represents the structure to be generated. The three-dimensional structure formation can be influenced in a directed manner by the deposition density of the nanofibers generated by means of an electrospinning process, which deposition density is adjustable through the accumulation time of the fibers.

In the method according to the invention, a conductive mold previously structured as a collector (template) is first placed on a standard conductive collector electrode under the capillary nozzle and then it is grounded together with the collector electrode. Given that the result is an intense interaction between the electric charges in the jet and the grounded mold, the jet extracted from the capillary nozzle can preferably be deposited directly on the grounded mold. Furthermore, the spiral-shaped line of flight of the jet upon approaching the template by means of the coulometric interaction between it and the grounded template or with a template with the opposite charge is strictly limited to the lattice rods in the template. Fibers are barely deposited, or no fiber is deposited, in the intermediate areas of the lattice rods in the template, where there is no conductive material (as in the openings of a mesh).

Consequently, the deposition position can be controlled with the simultaneous patterning of the jet.

With the electrospinning process according to the invention it is now possible to produce two- or three-dimensionally structured webs of polymer fibers both in any form and with a very high remote ordering with a controllable thickness and with a very high covering or depositing degree of the nanofibers by means of a mold (template) as a collector in a single work step. The method has not only the advantage that for the first time it allows producing multidimensional webs from nanostructures, which are joined to one another and therefore have a high stability. Also, it furthermore clearly requires fewer process steps and is therefore more favorable from the time and cost point of view and from a faster production point of view. It is therefore possible to open the necessary, special nanostructured webs to the mass market.

In order to consistently generate the structured or ordered webs, first the deposition of the nanofibers on a certain position or in an area in the collector electrode must be accurately controlled.

With the method according to the invention it is possible to locate in a controlled manner this deposition position on a smaller surface in the collector electrode. Furthermore, with a preferred embodiment method two- and three-dimensionally structured webs of polymer fibers in any form and with a very high remote ordering with a controllable thickness and with a very high covering or depositing degree of the nanofibers by means of a mold (template) as a collector can be produced in a single work step.

Compared with other processes, which require several process steps and consequently require a large expenditure from the time and cost point of view, the method according to the invention is simpler, faster, more effective and more economical.

However, unlike processes for generating oriented nanofibers by means of the electrospinning process (FIG. 3) described in the literature (D. Zhang et al., Adv. Mater. 2007, 19, 3664-3667 and D. Li, et al., Nano Lett. 2005, 5, 913-916), the method according to the invention is based on using a predefined conductive template, whereby the production of structured webs is allowed in a well-defined manner, having a high inner covering or depositing degree.

Unlike the state of the art, the deposition of spun fibers according to the invention takes place directly on the template used with high spatial precision when the predefined conductive template is used as a collector electrode. The generated structures in this regard exactly represent the predefined conductive mold (template).

The covering or depositing degree of the nanofibers is understood in the context of the invention as a measurement indicating how many of the spun nanofibers are deposited directly on the template and not between the hollow spaces. The covering or depositing degree of the nanofibers is preferably more than 95% in a single work step.

The conductive template which is located on a standard conductive collector electrode serves as a collector and is grounded together with the collector electrode. The polymer fibers are spun directly on the template (mold).

As was to be expected, the choice or the finish of the molds (templates) for patterning plays a decisive role. They must be flat and in all cases very conductive. The term flat is understood in the context of the invention as a two-dimensional mold, for example in the form of a net, lattice, etc., which can in turn be used for the desired patterning in a three-dimensional arrangement. Particularly, unlike the state of the art described above, the template according to the invention does not have any projecting elevation or sharp points in the area of the conductive areas of the template formed, for example, as lattice rods.

The intermediate spaces between the conductive areas of the template, which are configured as lattice rods, etc., on which the fibers must be deposited are empty, i.e., hollow spaces that are not filled.

An additional important factor for the patterning is the thickness of the mold. According to the invention, the thickness is in the order of magnitude of from 50 nm to 200 nm and from 200 nm to 500 nm for the generation of the represented microstructures with nanofibers, their separations between bundles of fibers ranging in size from 100 μm to 500 μm. Preferably, for the structures formed with nanofibers with separations between bundles of fibers from 100μ to 500 μm the thickness of the mold ranges from 500 μm to 2000 μm, and particularly for structures with separations between bundles of fibers ranging from 500 nm to 1000 nm the thickness of the mold according to the invention must range from 2 μm to 200 μm.

To obtain the fibers in an order of magnitude, the chaotic path of the jet must first be controlled in the most directed manner possible. Given that the electric charges are distributed throughout the jets coming out of the capillary, the paths of the jets can be controlled by means of external manipulation of the electric field. With a slight variation of the profile of the electric field, an influence on the deposition of the jets is clearly perceptible.

Based on this principle, a previously structured template, which generates a lack of homogeneity in the electric field, is additionally applied on a continuous conductive plate as a conventional collector electrode. Given that the operating force for arranging the fibers is the electrostatic interaction between the electrically charged jet and the conductive template, this interaction can be influenced in a directed manner by means of the shape of the templates.

The fibers are preferably deposited in the area of the structured template in the collector electrode given that the electric field intensity there has maximum values. Furthermore, the spiral-shaped line of flight of the jet upon approaching the template by means of coulometric interaction between it and the grounded template or the template with the opposite charge is strictly limited to only the lattice rods in the template. Fibers are barely deposited, or no fiber is deposited, in the intermediate areas of the lattice rods in the template, where there is no conductive material (as in the openings of a mesh).

Consequently, the deposition position can be controlled with the simultaneous patterning of jets.

In one embodiment of the invention, the template is used directly as a collector, such that the deposition of the jet is strictly limited to the conductive areas of the lattice rods in the template. Therefore, a deposition is advantageously made only in the area of the lattice rods and not in the intermediate area.

If the template is covered along the entire width at least once by the nanofibers, the spinning operation can be interrupted. Then the deposition layer of electrospun fibers is carefully separated from the template to obtain the self-supporting web, the structure of which corresponds to that of the template. The web which is generated in this regard is available for use or an eventual subsequent treatment. After the extraction of the web, the template can be used immediately for additional electrospinning operations.

According to the invention, the bundles of nanofibers are arranged, according to the previously structured template, in a highly oriented manner in one or two directions in a single work step with a very high degree of ordering the fibers without any additional modification or reconstruction for carrying out the electrospinning process.

If the fibers have been completely deposited on top of one another on the template, the charges remaining on the deposited fibers accumulate, the additional spun fibers being deposited without any limitation on the entire surface of the collector electrode, as in the case of a continuous plate in the standard electrospinning process. Therefore, the fibers can consequently be deposited be in a disordered manner, i.e., without a preferred orientation, between the lattice rods with a smaller thickness than the surface outside the template.

In the electrospinning process according to the invention, the nanofibers are intertwined by means of a repetitive adjacent and overlapping placement in the form of a three-dimensional web (nonwoven mat). The size and the shape of the hollow spaces between the fibers in such webs can be easily controlled such that applications as filter material, as protective clothing, as packaging material or in erosion protection and as a support matrix in biomedical applications and the transport and directed release of pharmaceutical preparations are conceivable.

Another object of this invention is the production of robust, structured, microporous and nanoporous webs from nanofibers arranged in oriented, electrospun bundles of fibers by means of a template.

The variety of the morphological characteristics resulting from the webs, which is based on the variation amplitude of the structure of the template, the polymer materials used and the modification possibilities of the self-supporting webs, opens the method according to the invention up to a large application potential.

Compared with the processes known until now, the method according to the invention presents the following advantages:

The structure of the electrospinning process has remained unchanged with respect to the conventional facilities, with the exception of the additional template, which is arranged on a conventional collector electrode (counter electrode).

The template can be previously structured and be easily and quickly finished for special applications.

The pattern formed from electrospun nanofibers corresponds to that of the template used.

The dimension of the webs can be freely adjusted to scale.

The up-scaling is therefore not limited by the dimensions of the web.

To obtain the self-supporting webs the structured deposition layers can be easily separated from the template.

The webs thus obtained can additionally be used for the construction of highly complicated structures.

The method according to the invention is characterized not only by its simplicity, comfort and high efficiency but also by the fact that self-supporting webs generated can be transported well and can thus be used for many applications.

The structured webs according to the invention are characterized, among others, by the following special mechanical and morphological properties:

The webs are for the most part microporous and nanoporous at the same time.

The webs can be produced at will according to the applications individually with more complex features.

In the resulting webs, the fibers are joined to one another by means of adhesive forces, whereby the webs together with the orientation of the fibers in the webs and the orientation of the microcrystallites, macromolecules, nanoparticles, etc. in the fibers themselves, have reinforcement properties, which decisively improve the handling of the webs during the additional processing.

An extremely notable property of the method according to the invention is that this technique allows the generation and orientation of spun fibers during the electrospinning operation in situ or simultaneously. The production of the nanofiber-based components or devices can thus be simplified.

According to the invention, the template can be made up of any conductive material which is in the form, for example, of wires and wire meshes or perforated metal grids, etc., of semiconductors or metal materials or in the form of fabrics made up of natural or chemical fibers, impregnated with a conductive agent to increase conductivity thereof. In this regard, there is no limit to the variety of structures of the templates produced by means of conventional micromanufacturing techniques.

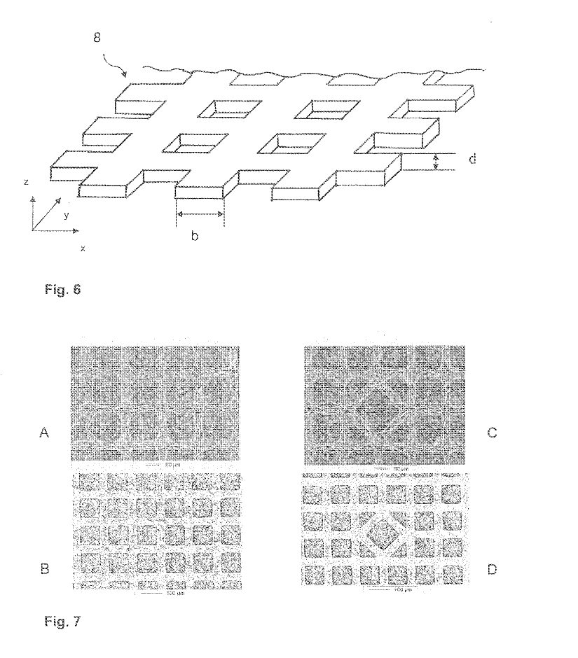

In one embodiment of the invention, in FIG. 6 the lattice rods of the template, which are made, for example, as wires, wire meshes or perforated metal grids, have a ratio of the width (b) of the lattice rods with respect to their thickness (d) of >1. This means that the lattice rods are wider than they are thick. The width (b) of the lattice rods characterizes in this sense the extension in direction x and/or y, whereas the thickness (d) of the lattice rods refers in this sense to the thickness of the material of the lattice rods in direction z. In this regard, it is particularly advantageous for the material to be essentially smaller in direction z than in direction x and/or y.

The method according to the invention allows producing webs of highly ordered nanofibers specifically for the application according to a customer's desire to best provide for use.

According to the invention, a polymer molten mass or solution is used for producing the structured webs of nanofibers, all the known natural and synthetic polymers, mixtures of polymers (polymer blends) and copolymers made up of at least two different monomers being used as suitable polymers provided that they can be melted and/or at least be dissolved in a solvent.

The polymer that can be used according to the invention can be produced according to processes known by the expert or it can be commercially obtained.

In this regard, polymers selected from the group consisting of polyesters, polyamides, polyimides, polyethers, polyolefins, polycarbonates, polyurethanes, natural polymers, polysaccharides, polylactides, polyglucosides, poly-(alkyl)-methylstyrene, polymethacrylates, polyacrylonitriles, latices, poly(alkylene oxides) of ethylene oxide and/or propylene oxide and mixtures thereof are preferred.

Especially the polymers or copolymers selected from the group consisting of poly-(p-xylylene); poly(vinylidene halides), polyesters such as poly(ethylene terephthalates), poly(butylene terephthalate); polyethers; polyolefins such as polyethylene, polypropylene, poly(ethylene/propylene) (EPDM); polycarbonates; polyurethanes; natural polymers, for example rubber; polycarboxylic acids; polysulfonic acids; sulfated polysaccharides; polylactides; polyglucosides; polyamides; homo- and copolymers of aromatic vinyl compounds such as poly(alkyl)styrenes, for example polystyrenes, poly-alpha-methylstyrenes; polyacrylonitriles, polymethacrylonitriles; polyacrylamides; polyimides; polyphenylenes; polysilanes; polysiloxanes; polybenzimidazoles; polybenzothiazoles; polyoxazoles; polysulfides; polyesteramides; polyarylenevinylenes; polyetherketones; polyurethanes, polysulfones, hybrid inorganic-organic polymers such as ORMOCER® by Fraunhofer Gesellschaft zur Förderung der angewandten Forschung e. V. Munich; silicones; fully aromatic copolyesters; poly(alkyl acrylates); poly(alkyl methacrylates); poly(hydroxyethyl methacrylates); poly(vinyl acetates), poly(vinyl butyrates); polyisoprene; synthetic rubbers such as chlorobutadiene rubbers, for example Neopren® by DuPont; nitrile-butadiene rubbers, for example Buna N®; polybutadiene; polytetrafluoroethylene; modified and unmodified celluloses, home- and copolymers of alpha-olefins and copolymers consisting of two or more of the monomer units forming the aforementioned polymers; poly(vinyl alcohols), poly(alkylene oxides), for example poly(ethylene oxides); poly-N-vinylpyrrolidone; hydroxymethylcelluloses; maleic acids; alginates; polysaccharides such as chitosans, etc.; proteins such as collagens, gelatins, their homo- or copolymers and mixtures thereof, are preferred.

In one embodiment of the method according to the invention, a solution of the aforementioned polymers is used for producing nanofibers, this solution being able to contain all the solvents or mixtures of solvents. In general a solvent selected from the group consisting of chlorinated solvents, for example dichloromethane or chloroform, acetone, ethers, for example diethyl ether, methyl-tert-butyl ether, hydrocarbons with less than 10 carbon atoms, for example n-pentane, n-hexane, cyclohexane, heptane, octane, dimethylsulfoxide (DMSO), N-methylpyrrolidinone (NMP), dimethylformamide (DMF), formic acid, water, liquid sulfur dioxide, liquid ammonia and mixtures thereof, is used. A solvent selected from the group consisting of dichloromethane, acetone, formic acid and mixtures thereof, is preferably used as a solvent.

In one embodiment, the mixing for the spinnable polymer solutions can be performed by stirring, under the action of ultrasounds or under the action of heat. The concentration of the at least one polymer in the solution generally amounts to at least 0.1% by weight, preferably 1 to 30% by weight, particularly preferable 2 to 20% by weight.

In the context of the invention, the corresponding polymer molten masses can also be used in addition to the polymer solutions provided that they are in liquid form. Hereinafter, the expression polymer solution will equally be used as a synonym for polymers which have been dissolved in solvents or which have passed on to liquid form by means of melting.

A large obstacle in the production of devices or components with the aid of nanotechnology is up-scaling the highly ordered structural unit. The movement or displacement of the template in direction x-y makes both the homogenization of the web layer thickness and the expansion of the dimension thereof largely possible. The thickness of the webs can be accurately adjusted by means of the deposition time and the shape of the webs by means of the structure of the template.

It is otherwise possible to easily apply any number of additional layers made up of different polymer materials on a web that is still on the template by means of electrospinning processes, whereby the generation of three-dimensionally structured, multilayer webs is allowed.

The minimum structure sizes of the webs that can be generated correspond to the diameter of the nanofibers which, according to the polymer and the process conditions of the electrospinning process, are in the range from a few nanometers to several micrometers.

The covering or depositing degree of the nanofibers in the method according to the invention, depending on the material and on the template, is in the range between 60 and 100%, which produces an increased mechanical load capacity of the webs.

The variety of the possible blends and functionalizations of the materials, the manipulation possibilities in the fiber structures, the specific modification of the application with color pigments, catalysts or metal, semiconductor or ceramic nanoparticles and the finish with healing drugs, enzymes or antiviral or antibacterial active ingredients, biological messengers (such as DNA, RNA and proteins) and the adjustable combinations of properties allow for a very wide range of application possibilities which cannot be achieved with conventional processes.

In one embodiment of the invention, all the known nanoparticles can be easily incorporated with different dimensions in the polymer molten masses or solutions before spinning and then be applied on the template together with the polymer as nanocomposite nanofibers. By incorporating nanoparticles, the advantages of the web structuring and the orientation of the fibers in the webs can be combined with the customized functionalities of the nanoparticles, whereby resulting in a number of fields of application.

In an additional embodiment of the invention, metals and/or semiconductors can be easily incorporated in the polymer molten masses or solutions with different dimensions before spinning as nanoparticles and then be applied on the template together with the polymer. Conductive nanofibers or nanostructures can thus be generated.

In an additional embodiment of the invention, active pharmaceutical ingredients can be easily incorporated in the polymer molten masses or solutions with different dimensions before spinning as nanoparticles and then be applied on the template together with the polymer.

When the webs produced according to the method of the invention are detached from the templates (self-supporting), they can be modified in a directed manner by means of different chemical and/or physical processes, in accordance with the respective case of application (irradiation with UV or gamma rays, treatment with plasma, impregnation, for example, with active pharmaceutical ingredients or catalytic precursors, etc.).

The structures according to the invention can further be subjected to surface modification with low-temperature plasma or by means of chemical reagents, for example, an aqueous hydroxide solution, inorganic acids, acyl anhydride, or halides or others depending on the surface functionality with organic silanes, isocyanates, anhydrides or halides, alcohols, aldehydes or alkylating chemicals with the corresponding catalysts thereof. By means of a surface modification, for example by means of coating or irradiating with high-energy radiation, the webs can obtain a more hydrophilic or more hydrophobic surface, which is advantageous in the case of use in the biological or biomedical field.

In an additional embodiment of the invention, to increase biocompatibility the surface of the nanofibers or of the webs according to the invention is modified by means of suitable processes, such as coating, adsorption, self-structuring, graft copolymerization, etc.

In one embodiment of the invention, ceramic nanofibers are produced by means of the electrospinning process according to the invention from a mixture of the polymer solution with a large number of suitable ceramic precursors. The ceramic precursors are preferably selected from the group consisting of Al₂O₃, CuO, NiO, TiO₂, SiO₃, V₂O₅, ZnO, CO₃O₄, MbO₃ and MgTiO₃.

A review of the processes for producing nanowires and ceramic nanofibers known up until now is disclosed in the literature (R. Ramaseshan at al. Journal of Applied Physics 102, 111101 (2007), Adv. Mater. 2004, 16, no. 14, pages 1151-1169).

In an additional embodiment of the invention, the fibers are enveloped, for example, by means of gas-phase deposition, sputtering, spin-coating, dip-coating, spraying, plasma deposition, sol-gel process or atomic layer deposition. The envelopment preferably takes place by means of gas-phase deposition or atomic layer deposition.

In an additional embodiment, the polymer is separated after enveloping the nanofibers. Suitable processes for separating the polymer are, for example, thermal, chemical, radiation-induced, biological, photochemical processes, and processes by means of plasma, ultrasonic, hydrolysis processes or by extracting with a solvent. Depending on the polymer material, the separation preferably takes place at 10-900° C. and at 0.001 mbar to 1 bar. The separation can take place completely or at a percentage of at least 70%, preferably at least 80%, particularly preferable at least 99%.

The high specific surface area is associated with a considerable capacity for the adhesion or the detachment of functional groups, absorption or adsorption of molecules, ions, catalytically active substances and different nanometric scale particles. Furthermore, the individual fibers and the fiber mats formed by them (webs) are particularly very suitable, due to their high specific surface areas combined with the high aspect ratio, high flexibility and strength, as reinforcement components in a polymer matrix for producing ultra-lightweight polymer composites.

In the electrospinning process according to the invention, the nanofibers are intertwined by means of the repetitive adjacent and overlapping placement in the form of a three-dimensional web (nonwoven mat). The size and the shape of the hollow spaces between the fibers in such webs can be easily controlled such that applications as filter material, as protective clothing, as packaging material or in erosion protection and as support matrices in biomedical applications and for the transport and directed release of pharmaceutically active substances are conceivable.

The method according to the invention described herein is a revolutionary technology for producing a controllable patterning of the electrospun fibers in a single work step, whereby the time-saving application of this method is allowed.

In one embodiment of the invention, the structured webs according to the invention are used as scaffolds in the field of tissue engineering or regenerative medicine. These scaffolds are used in the in vitro method for producing replacement tissues and organs to improve or maintain the function of diseased or damaged tissues. In this regard, the objective is to support a tissue defect only to the extent that it is needed during healing, such that new, healthy and functional tissue of the body is ultimately generated.

The support materials must comply with demanding requirements: they must be biocompatible, sterile according to the application or present long-term stability, or be biodegradable and have different flexibility. Furthermore, they must be porous so that the cells can penetrate them and in this regard still strong enough so that they do not tear during the first mechanical load.

The highly ordered scaffolds produced according to the method of the invention in different geometries and sizes comply not only with the objective of making a three-dimensional mold available for the cells and the extracellular matrix for the growth thereof, but they also assure enough mechanical stability to allow a particular appropriate organization of the tissue that is going to be cultured as well as an unhindered matrix deposition.

Due to the high porosity of the webs according to the invention with cavities (hollow spaces between the fibers) in the nanometric and micrometric range, the cells to be cultured occupy the webs in little time and with a high density (controlled cell growth). The nutrients can be easily transported to the cells and the metabolic wastes removed.

The bioresorbable polymers are used in a reinforced manner due to the different mechanisms of degradation and of the adjustable degradation times associated with them in medicine. When the scaffold materials are made up of such bioresorbable polymers, the generated tissue or cell bandage can be transplanted together with the scaffold. The polymer materials slowly break down in the body due to their biodegradability, the remaining tissue of the body gradually adopting the function of the tissue or organ without requiring another surgical intervention.

The fibers can also be provided during the electrospinning process or by means of a subsequent modification of the webs with different messengers, for example growth factors (attraction of cells, stimulation or acceleration of the growth of the added cells), or medicinal products, for example antibiotics and antiseptics, for the purpose of the directed release of pharmaceutical preparations in the organism after the implant.

The term tissue in this sense means an accumulation of cells of an individual organism which are optimally specialized for performing a specific task. Particularly cardiovascular tissues or contractible, mechanically robust muscles have oriented cell morphology with a higher density. To culture such functional tissues, it is desirable for the scaffolds to not only support cell-to-cell interaction but they must also be available for the orientation of the cell, imitating the original cultured tissue structures.

It was shown in the literature that the cultured cells can be made to proliferate on the scaffolds, the fibers being oriented one-dimensionally, preferably the direction of the fibers (C Y. Xu, et al., Biomaterials 25: 877 (2004); C H. Lee, et al., Biomaterials 26: 1261 (2005)).

The webs produced with the method according to the invention comply with the requirements for one-dimensional and two-dimensional structures for producing especially those types of tissues. They offer not only basic imitation scaffolds for natural, nanometric scale, extracellular matrices, but they also form a defining architecture necessary for guiding cell growth or development. The orientation that can thus be achieved from the cells in a controlled, one-dimensional, two-dimensional and three-dimensional architecture has a decisive significance for cell differentiation, proliferation and functional longevity (life).

The capacity of the method according to the invention for generating large amounts of highly oriented fibers offers the possibility of performing clinical cell behavior studies, such as, for example, gene expression and cell interaction, tissue toxicology, etc., depending on the orientation of the fibers.

In an additional embodiment of the invention, the structured webs according to the invention are used for producing special plasters for blood clotting.

Ideal dressings for wounds must maintain, in addition to their function of support and preventing the penetration and proliferation of microorganisms, above all else the moist physiological microclimate and thereby favor healing. Gas and water vapor permeability must also be assured given that an unchanged epithelization needs a sufficient amount of dissolved oxygen in the wound secretion. The formation of scabs must further be prevented because while they do protect against external influences, they also agglutinate the secretion and thus block the migration of the new cells formed. Special embodiments also reduce the formation of scars.

Based on the method according to the invention, a new generation of wound plasters is developed made up of biocompatible and resorbable nanofibers, whereby healing is considerably accelerated.

A particularity of the electrospun fibers is their nanoporous surface structure, the nanopores of which soak up the exudate of the wound and block out germs and tissue and tissue residue in an effective manner. However, they also encourage maintaining a moist medium which favors healing.

In an additional embodiment of the invention, the nanofibers are loaded with different types of pharmaceutical substances, such as, for example, growth factors (attraction of epithelial cells, stimulation or acceleration of the growth of the added epithelial cells) or medicinal products (antibiotics, antiseptics, particularly medicinal products inhibiting pain and bleeding which are suitable for topical application, to create the prior optimal conditions for the fast healing of wounds.

In an additional embodiment of the invention, the plaster for wounds loaded with messengers biologically degrades in gradual manner during the healing process, whereby painful bandage changes, which in turn also partially causes the detachment of the new tissue formed to a great extent, can be eliminated. Furthermore, the plaster for wounds can administer one or several medicinal products according to patient requirements to the wound site during a specific time period.

With the technology according to the invention the wound plasters can both be produced specifically for the patient in different sizes and configurations and be loaded for a specific condition (diabetes, occlusive arterial disease, chronic venous insufficiency, among others) with special active ingredients. The wound plasters therefore allow a time-saving, easy to perform and cost-effective wound healing therapy.

In an additional embodiment of the invention, the webs produced according to the invention from nanofibers are used as support tubes for regenerating blood vessels, the esophagus and nerves. Vascular lesions or aneurisms, which were treated up until now by means of coiling (endovascular occlusion of the aneurism), for example, can thus be satisfactorily treated. The use of the support tubes according to the invention as endoprosthesis is also envisaged. In an improvement of this embodiment, improved healing is possible by loading the support tubes according to the invention with pharmaceutically active substances by means of the in situ release thereof. The necessary doses of the applied substances could thus be further reduced, avoiding systemic application.

In an additional embodiment, the support tubes produced according to the invention are produced from biodegradable substances. Therefore there is a single temporary incorporation of foreign bodies in the corresponding tissue section, whereby preventing possible subsequent rejection reactions.

In an additional embodiment of the invention, the biodegradable support tubes according to the invention are loaded with pharmaceutically active substances. Due to the biodegradability the constructs of this type perform a deposit function, the active ingredients being released over time into the surrounding tissue and the deposit itself experiencing degradation at the same time. Active ingredient deposits which can be used in a directed manner at the site of action can therefore be produced using minimally invasive techniques, without a subsequent removal being necessary.

In an additional embodiment of the invention, the webs produced according to the invention from nanofibers are used for implant surface modification. The immune response and its associated danger of implant rejection can be reduced or minimized by means of the corresponding surface functionalization. It is possible for the cells of the body to occupy the implant by means of a suitable coating with proteins, such as extracellular matrix proteins, signaling proteins, cytokines, etc.

In an additional embodiment of the invention, the implants are provided with an antimicrobial coating by applying biocompatible and biofunctional electrospun nanofibers on the implants. Possible inflammations caused by germs are thus prevented. Typical examples of this are webs with embedded TiO₂ as a photocatalytic coating for self-sterilizing and biofiltration applications. In addition, MgO and ZnO nanoparticles are used as effective disinfecting agents in dyes for inner walls.

In one embodiment of the invention, different inorganic materials containing metals are used in the fibers as antibacterial agents; such as, for example, silver, copper, zinc and other antibacterial metals as inorganic disinfecting agents. The antibacterial agents are continuously released from the webs produced by means of the method according to the invention into the environment over a long time period. Compared with other conventional methods of administration, the release of disinfecting agents by means of the web produced with the method according to the invention offers higher value with respect to heat resistance, safety and durability.

In an additional embodiment of the invention, the webs produced according to the invention from nanofibers are produced as porous membranes and are used as a temporary skin graft. In this regard it is advantageous for the webs according to the invention to be prepared from biodegradable substances.

In an additional embodiment of the invention, the webs produced according to the invention are used as support tubes in nerve regeneration. The webs according to the invention are coated with suitable signaling substances, whereby nerve cell proliferation throughout the web is favored. These coated webs are then used in the area of the broken nerve connection. Adjacent nerve cells are stimulated by the signaling substances applied on the web to proliferate towards the web. New neural connections are formed as a result, whereby reconnecting the transmission of nerve impulses that had been interrupted.

In one embodiment of the invention, the structured webs according to the invention are used for producing ultra-lightweight polymer composites.

Due to the high specific surface areas of the structured webs according to the invention combined with the high aspect ratio, high flexibility and strength of the fibers, said fibers especially are very suitable as reinforcement components in a polymer matrix for producing ultra-lightweight polymer composites.

In one embodiment of the invention, the structured webs according to the invention are compacted by means of a hot-compaction process in established process conditions (pressure, temperature), without destroying web structuring and orientation for producing polymer nanocomposites.

The composites reinforced with the webs produced with the method according to the invention allow a customized combination of the properties of the materials; on one hand, sufficient voltage transmission through the matrix-fiber boundary surface is assured, but on the other hand tolerance to damage is increased (stopping tears, deviating tears).

Possibilities of varying the properties result from a modification of the morphology of the web, i.e. of the thickness, distribution and orientation of the fibers.

Due to the size of the fibers, the compacted webs show a more intense polymer-fiber interaction in the boundary layer of the fibers with respect to the matrix. With such surface hardening, the corrosion resistance, fatigue strength and impact strength of the layers, i.e., essential properties for use, can be improved. Increased microporosity and nanoporosity of the web further offers better grip.

Unlike fiberglass composites, these novel polymer balanced property profile (for example strength, rigidity and tenacity) with a reduced specific weight and are therefore open to a wide range of application possibilities.

The optical properties of the resulting nanocomposites, such as the high transparency of the composites compared with the unmodified matrix materials are also very essential for the use of the webs according to the invention. The transparency is brought about because the diameter of the nanofibers is considerably less than the wavelength of visible light.

The ultrafine fibers with diameters of up to a few nanometers can further be modified without any problems with different nanofillers, such as, for example one-dimensional, carbon nanotubes, two-dimensional layer silicates and three-dimensional nanoparticles. In comparison, the challenge in conventional processes lied in homogenously dispersing the nanoparticles in the fibers, preventing agglomerates and therefore voltage concentrations in the matrix material in the case of a charge.

Due to the extremely high shear force during the electrospinning operation, the nanoparticles originally arranged in a disordered manner are ordered with a virtually parallel arrangement in the nanofibers. Certain properties (strength, diffusion barrier, flame retardance) are thus improved.

The percentage of nanoparticles in the compact nanocomposites is usually 0.1-5% by weight (weight percent) and is therefore very low compared with conventional mineral loads. The weight percent of the nanoparticles in the nanofibers is often clearly below 0.001% by weight.

In one embodiment of the invention, the webs according to the invention are modified with nano-layer silicates. These polymers modified with nano-layer silicates, for example montmorillonite, hectorite and saponite, have improved properties with respect to resistance to UV rays and to heat, reduced inflammability and gas permeability and increased biodegradability in the case of the biodegradable polymers.

In an additional embodiment of the invention, carbon nanotubes (CNT) are dispersed in the polymers. Composites characterized by a higher mechanical strength and higher thermal and electrical conductivity are generated by dispersing carbon nanotubes (CNT) in the polymers.

In an additional embodiment of the invention, the webs according to the invention are used as filtering means.

The electrospun webs generally have the consistency of typical porous membranes, their porosity reaching the order of magnitude of 60 to 80%. The high pore density with an adjustable pore size (microporosity and nanoporosity) result in applications as filter material (liquid and gas filtration, molecular and bacterial filtration, clean room technology, climate control installations).

By means of the production method according to the invention, the membranes have special surface characteristics as a result of which their physically and/or chemically active substances are immobilized in the structures in the form of fibers. In order to also deposit the small particles in the most secure manner, the pores must be as small as possible with small pore diameter distribution amplitude. Given that the flow resistance must be as small as possible, large porosity or a large flow surface area is preferred.

Due to the large surface area of the nanofibers, the webs according to the invention have a high adherent dirt particle capturing capacity with a high permeability of the substance to be fixed. Compared with conventional small-pore filtering means, they have the advantage of a clearly smaller complete pressure loss with the same or higher capturing capacity and therefore extend the service life of the filter. The extension of the service life is a factor which reduces filter-related operating costs.

The probability of retaining a very fine nanofiber particle in the air current increases simultaneously with the number of nanofibers. In the case of the webs according to the invention, a high percentage of nanofibers which also have very high porosities almost completely retain even the finest particles and fine powder in the filtering means.

The fine network structure similar to a fabric with very small intermediate fiber spaces allows, in the case of the webs according to the invention, retaining particles with a very high depositing degree, however the liquid and/or gases can pass through unhindered.

The webs according to the invention as filtering means are consequently characterized by an excellent balance between deposition performance, air permeability and service life.

In addition to complying with the deposition function, to assure a sufficiently long service life for the technical use of nanofibers in filters different mechanical and physical aspects such as modulus of elasticity, tensile strength, limiting bending stress, wear resistance, moisture absorption, cold flow, temperature resistance, thermal conductivity, electrical resistance, light resistance, weight, among others, must also be taken into account.

Although the nanofibers distributed in a disordered manner between the highly ordered areas of the webs are decisive for the filtration of the smallest particles, the oriented nanofibers in the form of a lattice contribute to the tensile strength of the filtering means according to the load. The nanofibers forming the structure further increase the cracking resistance of the filtering means.

A high deposition performance is thus combined with greater permeability and with a mechanical stability that is as great as that of the medium.

The webs according to the invention are used in challenging industrial filtration under the toughest conditions and in special filters for heavy vehicles, i.e., in applications in which a very small filter weight and high permeability and/or large specific surface area of the filter are required.

By means of the method according to the invention the structuring of the web can be controlled such that webs exactly adapted to the requirements of the specific separation processes are constructed.

To modify the surface properties, i.e., to modify the electrical conductivity or use properties, the webs can also be provided with finishes, these coatings having only a limited fatigue strength.

The different webs can further be compacted with one another without destroying their structure. For example, a less mechanically stable, fine web of less thickness for optimizing deposition can be combined with a mechanically robust support web for optimizing the load capacity.

The obstruction of the filtering means can be counteracted by means of backwashing, spraying, stressing with ultrasounds, lixiviation, among others. The simpler the configuration of the pore structure of the filtering means, the easier it will be to prevent their permanent obstruction.

The main advantage of this technology, in addition to the price advantage, lies in being able to develop and produce client-specific products in which the gradient between coarse and fine porosity can be freely adjusted within a broad spectrum.

The advantages of this technology are a clearly improved filtration efficacy, a clearly improved service life, less production expenditure and consequently lower costs, an adjustable nanofiber and coarse fiber gradient, the protection of the integrated nanofibers against mechanical damage and less raw material use.

In an additional embodiment of the invention, the nanofibers and/or the webs produced according to the invention are used for the coating and/or as a component of textile materials.

It is common practice to generate specific properties of the synthetic fibers directly by means of the production method, given that technical textile materials must meet special requirements according to their different applications. The properties of the fibers in the webs according to the invention can be adjusted in a directed manner according to the requirement.

The particularity of the webs according to the invention is based on their very large surface area. Furthermore, due to the well-defined orientation of the nanofibers they have increased tensile strength and reduced gas permeability, whereby they are suitable for very different applications.

By means of introducing a wide range of additives (for example color pigments, drops of latices, with catalysts, enzymes, drugs, semiconductors or metal nanoparticles, etc.) in or on the fibers, new finished textiles will be developed which lead to generating new textiles products with essentially improved properties or properties that have not been described until now or properties that allow combinations of functions (antibacterial, self-cleaning, conductive, antistatic, ultraviolet (UV) radiation protection, flame protection, thermal insulation and many more) which are based on the effects of the nanostructures.

In one embodiment, the webs according to the invention are applied in the textile industry as special textile materials with excellent thermal insulation properties, as protective clothing to minimize air impedance, textile materials with high adherence efficacy for nanoparticles and antibiochemical gases and for photochromatic or thermochromatic clothing by means of incorporating color pigments in the nanofibers.

When the fibers are metalized in a textile material or their conductivity is increased, body functions such as heart beat, temperature or blood pressure, for example, can be measured. This and a high wear comfort are assured with a fine, nanometric metal coating.

A simple possibility in principle for increasing the electrical conductivity of the nanofibers is to incorporate conductive materials in the form of particles finely distributed in the polymer matrix.

In one embodiment of the invention, conductive materials in the form of particles finely distributed in the polymer matrix are incorporated for protection against electrostatic discharges in protective work clothing. Protection against electrostatic discharges is indispensable in many occupational safety fields. The results are thin nanometric metal layers, deposited in the process which increase the conductivity of the polymers several orders of magnitude. Metals (such as gold, silver, aluminum, iron, copper, nickel), carbon (in the form of soot, graphite or currently carbon nanotubes) or conductive polymers (polyaniline, polypyrrole, polyethylenedioxythiophene) are used as conductive materials. Consequently, the fibers can be used as electrical conductors in the field of antistatic agents.

In an additional embodiment of the invention, the incorporated silver particles or the silver coatings deposited on the nanofibers act as antibacterial agents. The fibers enveloped with silver in the special washing for patients with neurodermatitis provide, for example, an improved clinical picture. The webs mixed with silver can further be used in the public health sector to control the propagation of antibiotic-resistant bacterial strains. Operating room sheets and other textile implements prevent the spread of infections as a result of a silver finish, given that the bacteria are killed in one hour.

In an additional embodiment of the invention, the textile materials according to the invention for medical applications and for applications in the leisure/wellness field are spun with active ingredients or fragrant substances (cyclodextrins or iodosobenzoic acid and different deodorants). The nanometric scale deposit structures can bond to the fragrant molecules and be released again in the following washing.

In an additional embodiment of the invention, the elimination of bacteria can also be used to control the smell of sweat in sports clothing given that the smell of sweat is generated by bacteria. Since the pores in a web according to the invention are essentially smaller than a drop of water, the web is very impermeable to water and to the wind. However, it allows the passage of body moisture as water vapor. The webs according to the invention are also breathable and therefore allow evacuating (diffusing) the evaporated sweat, which is very important for regulating body temperature. If athletes sweat excessively when exerting high efforts, they will feel a body chill perceived as unpleasant. This so-called post-exercise chill effect can be prevented by means of nanostructuring the fibers because their capillary effect provides a fast evacuation of the sweat.

The textile materials according to the invention allow regulating the temperature and the microclimate which are formed between the surface of the skin and the layers of clothing closest to the skin. This microclimate is most significant in relation to the wear comfort.

Furthermore, the textile material according to the invention also advantageously presents the self-cleaning principle similar to that of the leaf of a lotus plant and many insect species. No water and/or dirt can penetrate the textile materials due to high pore density in the web structure. As a consequence of the nanostructuring, both water and dirt remain on the surface of the web. The webs according to the invention therefore provide excellent protection for the textile materials against dirt. The textile materials according to the invention are further characterized by highly effective, long-term water impermeability with breathing activity at the same time.

Important product properties which can be developed by means of the method according to the invention are, for example, easy to clean properties, protective layers (barrier layers, sliding layers, etc.), the directed arrangement of switchable nanolayers or nanostructures, electrical conductivity, catalytic efficacy, catalytic self-cleaning, electromagnetic shielding, substance-specific binding and filtration properties, controlled release of active ingredients and improved flame resistance, elasticity and processability.

In an additional embodiment of the invention, the textile materials according to the invention are used in car seat covers, climate control air filter installations, in the form of awnings and cloth coverings in buildings or as operating table covers in hospital facilities.

In accordance with the method of the invention, advantageous polymer blends which can be spun to yield a complex material when blending two or more different webs and which structurally adapt to one another can be produced to generate structural or functional properties which the individual components alone do not have.

In one embodiment of the invention, the web supports according to the invention are used for catalysts, whereby they can be used for catalytic processes.

The webs according to the invention made up of nanofibers have excellent properties, particularly a large specific surface area and high liquid and gas permeability. Furthermore, the structuring of the fibers in the micrometric and nanometric areas forms a stable web and allows easy handling.

The catalyst is immobilized exclusively in the nanofibers by means of electrospinning a mixture of the polymer matrix with the catalyst or a catalyst precursor. In the resulting nanofibers the catalysts are encapsulated in the nanofibers, the web acting as a semipermeable membrane. This immobilization allows short diffusion paths and therefore a reduced limitation of substance transport. Accordingly, the catalyst-immobilized nanofibers show shorter reaction times than conventional films do, but for that purpose they also show more reduced sensitivity due to the lower contact resistance, and in a secondary manner this leads to increased activity of the immobilized catalyst (fast reaction time).

Compared with conventional thin films, the catalyst concentration can furthermore be considerably reduced by means of a molecular dispersion combined with the nanostructuring of the web. The reduced residual concentration in the end products can thus be maintained.

In applications in medicine, pharmacy, electronics and optoelectronics, the synthesized products must be present especially with a higher degree of purity. In other words, the catalyst must be able to be easily separated from the product to a greater extent. Immobilization in the nanofibers allows such a recovery of the catalyst from the reaction medium in a very high percentage.

The range of catalysts that can be used for the webs according to the invention is very broad, starting with metals, including gold, silver, osmium, ruthenium, palladium and platinum, continuing with inorganic compounds such as, for example, semiconductors (lead sulfide, cadmium sulfide, titanium dioxide, zinc oxide and others) and zeolites, and up to biomolecules or enzymes.

These novel catalysts combine simple handling, general applicability and high activity. The webs functionalized with different catalysts can be used in chemical synthesis.

For use in nanoelectronic circuits and components, electronically active catalysts and materials can be deposited on the nanofibers according to the invention with aid of PVD processes or sol-gel coating processes.

Improved detection properties are achieved in the case of webs according to the invention by means of the finer structuring of the nanofibers which can be achieved according to the invention. In addition to a considerably faster reaction time based on the short paths between the catalyst and the reaction medium, the webs according to the invention used as detection materials can detect metal ions and vapors in a manner that is two to three orders of magnitude more sensitive than thin film sensors. The nanofibers according to the invention can thus be used for developing gas detectors.

In an additional embodiment of the invention, novel, highly active biocatalysts are obtained for reactions in organic solvents by means of adding enzymes during electrospinning. Due to their high porosity, the webs according to the invention are envisaged for use in biosensors and biofuel cells.

In an additional embodiment of the invention, the nanofibers according to the invention are used as part of optoelectronic components. It was shown that the electrospun nanofibers made up of conjugated polymers have excellent photoluminescence and electroluminescence as photovoltaic and of nonlinear optical properties. The nanofibers can therefore be considered as promising materials for optoelectronic components.

The conjugated polymers are an important class of materials due to their semiconductive properties. Similarly to inorganic semiconductors, very high electrical conductivities can be achieved by means of doping so they are also referred to as “synthetic metals”.

The applications of the materials according to the invention range from materials for organic light-emitting diodes, nonlinear optics and organic polymer lasers, to polymers for photovoltaic applications (solar cells), to semiconducting polymers for polymer electronics (field-effect transistors), computer chips and display technology.

Compared with conventional semiconductors, the electroluminescent polymer materials are, especially in the development of large surface area displays which at the same time can be bent or rolled up, a real and cost-effective alternative to conventional cathode ray displays and liquid crystal displays (LCD).

They can otherwise lead to the development of monochromatic color displays with high light intensity, for example for mobile telephones or computer displays which, unlike the LCD technology used until now, have several clear advantages, such as lower power consumption with a higher light intensity at the same time, and better contrast or the independence of the viewing angle.

The conjugated polymers are especially versatile given that a simple and fine adjustment of their properties (color, quantum efficiency) is possible by means of modifying the structure. In this regard, the nanostructured polymer materials invoke increasing greater interest as an active or passive component in electronic components.

The one-dimensional fibers of conjugated polymers represent novel, cost-effective and flexible components combining electronic, optical and mechanical properties, which are potentially suitable for use in nanometric scale, function electronic and optical components.

In one embodiment of the invention, a light-emitting diode is made up of semiconducting polymer nanofibers. It is a promising, favorable and very small light source.

In an additional embodiment of the invention, the webs according to the invention based on electroluminescent nanofibers are used in lasers, flat displays and luminaires.

The color of the webs according to the invention, for example red, yellow and green, can be adjusted by using the suitable polymer semiconductors. The emission of the electrospun fibers can additionally be easily adapted from a visible wavelength to near infrared wavelength (NIR) by incorporating active molecules (chromophores).

In an additional embodiment of the invention, the nanofibers which emit light from the near-infrared range are used for applications in communication networks, biosensor technology and photonic technology-based diagnostics.

The emission produced by the light-emitting electrospun nanofibers is limited to the nanometric scale due to fiber size. However, due to the increased charge mobility and the very high-speed charge and discharge rate in the nanofibers, it leads to an attractive property for applications in the field of sensors, where a highly localized molecule excitation is required, for example for DNA and protein scanning.

In an additional embodiment of the invention, the nanofibers according to the invention are used for sensor systems (chemical resistor) with greater sensitivity and selectivity due to their extremely high, intrinsic specific surface area.

The acids, bases, oxidants, anions, cations, inorganic and organic gases can have an effect on the electrical conductivity of the webs according to the invention.

In an additional embodiment of the invention, the nanofibers according to the invention made up of conjugated polymers are used in field-effect transistors. Technologically speaking, field-effect transistors are important additional components based on conjugated polymers given that they form the basic module in logic circuits and display switches.

The webs according to the invention therefore open up the possibility of the high performance and cost-effective production of completely organic photon systems based on coherent emitters.

In an additional embodiment, the webs according to the invention are used in solar cells. The webs according to the invention subjected to electrospinning are used in solar cells as a solution of the semiconducting polymers with the acceptor molecules, for example fullerenes (C60). A light-absorbing web according to the invention is thus generated, in which the boundary surface in the polymer and the electron conducting acceptor phase is distributed by the volume of the layer, the electrons generated quickly passing through the light of the polymer to the acceptor molecule and traveling the necessary path for removing the charges towards the electrode as quickly as possible.

The fundamental advantages of a solar cell based on the webs according to the invention with respect to conventional webs are the lower manufacturing costs due to less expensive production technologies, high current efficiencies by means of the increased specific surface area and flexibility as well as easy handling.

In an additional embodiment of the invention, the organic photovoltaic systems produced based on the webs according to the invention are configured such that they are rollable.

In an additional embodiment of the invention described above, the organic photovoltaic systems produced based on the webs according to the invention are integrated in chip cards and textile materials.

In an additional embodiment of the invention, the webs according to the invention made up of polymer semiconductors are used as protection against electrostatic discharge, corrosion protection and electromagnetic interference shielding.

In an additional embodiment of the invention, nanomagnetic particles are added to the polymer molten mass/solution before spinning. The nanomagnetic particles are of great interest for many applications due to their numerous exceptional properties, such applications ranging from high-performance data storage and catalysis, to biotechnology/biomedicine; for example for electrochemical biosensors and bioseparators, for detecting DNA, RNA, cells and proteins, for medicinal product and gene transport or controlled release systems, as a contrast agent for nuclear magnetic resonance imaging, hyperthermic treatment for cancer tumors and cells.

In the processes according to the invention, nanomagnetic particles with a large number of different compositions and phases are used; for example with Fe₃O₄ and γ-Fe₂O₃, pure metals such as Fe, Ni and Co, spinel-type ferromagnets such as MFe₂O₄ (M being a metal such as Mn, Co, Ni, Cu, Zn, Mg, Cd, etc.) as well as alloys such as CoPt₃ and FePt, and magnetic nanocrystals such as Cr₂O₃, MnO, Co₃O₄ and NiO.

Regardless of the application of the nanomagnetic particles in the nanofibers, particle stability maintenance over a long time period without agglomeration or precipitation represents increased difficulty.

By means of the electrospinning process according to the invention, such stability can be easily achieved by immobilizing or encapsulating the nanoparticles in the nanofibers. In the case of the webs according to the invention, the polymer matrix serves as an protective envelopment not only for protecting the nanomagnetic particles against oxidation and erosion or decomposition, but also for the additional functionalization, for example with catalytically active species, active ingredients, specific binding sites or other functional groups.

In an additional embodiment of the invention, the nanomagnetic particles are used in the catalysis and in the separation of biological species.

The ferromagnetic nanoparticles, the size of which is below a critical value, usually having a diameter of approximately 10 nm, show superparamagnetic behavior, which means that they can be magnetized with an external magnetic field and then be immediately redispersed after removing the magnet.

These properties make the superparamagnetic nanoparticles extremely interesting for a wide range of biomedical applications given that the risk of the formation of agglomerates at room temperature is discarded.

Such magnetic behavior in the form of a simple connection/disconnection is a special advantage of the magnetic separation.

Especially in the case of liquid phase catalytic reactions, such small, multifunctional, magnetically separable particles have enormous potential because the webs according to the invention combine the advantages of high dispersion, high reactivity and easy separation capacity.

The webs according to the invention containing such nanomagnetic particles can be suitable as magnetically switchable bioelectrocatalytic systems for effective, fast and simple separation and reliable catalyst, radioactive residue, biochemical product, gene, protein and cell capture.

The accumulation and subsequent separation of the biomolecules with little concentration, such as, for example, target DNA/mRNA molecules with the webs according to the invention is of great interest for the diagnosis of diseases, in gene expression studies and in studying genetic profiles.

In one embodiment of the invention, the webs according to the invention are made up of biocompatible polymers with nanomagnetic particles to which pharmaceutically active ingredients are bound. They are used as magnetic field-controlled drugs (magnetic-drug targeting).

In an additional embodiment of the invention, nanoparticles are used in addition to the pharmaceutically active ingredients at the same time as a contrast agent. In addition to the delivery of targeted magnetic field-controlled active ingredients, this also results in a possibility of real-time control by means of nuclear spin tomography.

The webs according to the invention can transport a high dose of the active ingredient and thus provide a high local active ingredient concentration in situ. Toxicity and other side effects due to a high systemic active ingredient dosage in other parts of the organism are thus prevented.

In an additional embodiment of the invention, the nanomagnetic particles are used in hyperthermic treatment. This treatment is considered a complement to chemotherapy, radiation therapy and surgical interventions in cancer treatment. The idea of using hyperthermia by magnetic induction is based on the fact that heat is produced due to the loss of magnetic hysteresis (Néel and Brown relaxation) when the nanomagnetic particles are exposed to an alternating magnetic field.

If a web according to the invention is exposed to an alternating magnetic field, these superparamagnetic particles are converted into intense heat sources that destroy tumor cells given that these cells are more temperature-sensitive than healthy cells are.

In an additional embodiment of the invention, purely magnetic fibers are produced by means of spinning polymers with suitable precursors and the subsequent thermal treatment of the spun fibers. The webs according to the invention made up of magnetic fibers are used for high-density data storage media, magnetic logic junctions, spintronic devices, magnetic sensors and magnetic composites.

In an additional embodiment, metal, ceramic nanofibers and their hybrid nanoparticles are produced by means of electrospinning processes or directly from the corresponding precursor materials or in event that they cannot be submitted to electrospinning, from a sufficiently viscous polymer solution containing the precursor materials, the polymer acting as a support.

The resulting organic-inorganic precursor nanofibers can be structured or oriented according to the invention with aid of a suitable template. The webs made up of these fibers are then thermally treated (for example in a furnace at a temperature that leads to the degradation of the matrix polymer, for the removal or pyrolytic sublimation of the polymer component directly and without any problems). By means of the associated pyrolysis of the matrix polymer, the polymer components are effectively separated such that purely inorganic nanofibers made up of metals, ceramic materials or hybrid metal/ceramic materials are obtained.

The webs according to the invention made up of numerous nanofibers, such as, for example, metals; Cu, Fe, Ni, Co, Pd and Fe₃O₄, etc., ceramic; ZnO, TiO₂, NiO, CuO, MgO, Al₂O₃, are thus produced. Otherwise, the fibers can also be made up of cobalt nitrate and cobalt dinitrate, iron nitrate and iron trinitrate (Fe(NO₃)₃*9H₂O), nickel(II) acetate tetrahydrate or palladium acetate, etc. Based on this principle, carbon nanofiber webs can also be generated from electrospun polyacrylonitrile nanofibers.

In an additional embodiment of the invention, due to their very large specific surface area with excellent mechanical stability, the nanostructured ceramic webs according to the invention are used in the hot gas filtration and in generating electricity from exhaust gases from machines.

In an additional embodiment, the nanostructured ceramic webs according to the invention are used in all the applications in which conventional ceramic materials have been used up until now. For example, the nanostructured ceramic webs according to the invention are used in catalysis, fuel cells, solar cells, membranes, hydrogen storage batteries, structural applications, applications requiring high mechanical rigidity, for biomedical applications, such as tissue culture/tissue technology (tissue engineering), biosensors, etc.

In one embodiment of the invention, nanostructured ceramic oxides are applied further due to their special electronic properties in the field of nanoelectronics, sensors technology, resonators and in optoelectronic and magnetoelectronic devices.

The sub-micrometric particle capture performance can be increased by means of the increased specific surface area of the webs according to the invention, such that a new generation for gas sensors can be generated in climate control and medical applications.

In an additional embodiment of the invention, the polymer webs according to the invention are used as a template for producing the large surface area, self-supporting nanostructured webs made up of nanotubes, these webs having at least one inorganic component.

In this regard, the web according to the invention is first covered with a so-called lining material. Different techniques are provided for applying the lining material on the fibers depending on the material used. Gas-phase deposition (chemical vapor deposition—CVD), sputtering, spin-coating, sol-gel process, dip-coating, spraying, plasma deposition or atomic layer deposition (ALD) are mentioned by way of example.

In one embodiment of the invention, the depositions preferably take place from the gas phase. Therefore, not only are a layer with a very uniform thickness around the fibers and a very accurate reproducibility of the surface topology of the fibers of the template obtained, but impurities, for example due to solvents, are also prevented.

ALD is particularly suitable, in which process, unlike CVD, the growth of the layers is cyclical. The self-controlling growth mechanism in ALD facilitates the film thickness control and control of the composition at the atomic level, which allows deposition on large complex surfaces. The polymer matrix is pyrolytically separated after the deposition of the inorganic phase on the nanofibers.

Complex structured webs can thus be reproduced quickly and easily with inorganic materials. Depending on the precursor materials available, self-supporting webs made up of metals, ceramic and hybrid nanotubes can be produced. The geometry of the tubes generally offers considerable advantages given that the nanotubes can be used both as ducts and as microcavities or microcapsules.

The webs according to the invention with accurately defined nanometric scale walls form easy-to-handle nanostructured systems with an extremely large surface area which, compared with the conventional web systems, can be used advantageously for example in catalysis or in sensors.

The properties of the webs made up of nanotubes with at least one inorganic component can be custom-adapted by means of the functionalization of the walls of the nanotubes to the respective case of application.

The morphology surface of the nanofibers which can be adjusted in a directed manner by means of phase transitions or phase separation processes is reflected in nanorugosity or nanoporosity of the walls of the tubes. The surface area of the wall of the tubes is thus increased again, which is advantageous for many applications, for example in catalysis, substance separation or sensor technology.

In one embodiment of the invention, the additional nanopores can be used as containers for the molecule, messenger and active ingredient transport.

The successive coating of the wall with different materials increases the spectrum to multilayer nanotubes and also multicomponent systems and composites with a defined composition which can be formed to yield nanotubes.

In an additional embodiment of the invention, the nanofibers according to the invention can be formed by means of an additional coating with one or several precursor materials to yield hybrid nanotubes with a core-enveloped morphology.

The nanotubes according to the invention or the webs made up of the nanotubes can be used in a versatile manner.

In one embodiment of the invention, the nanotubes or the webs made up of the nanotubes are used in the medical and pharmaceutical field (tissue engineering, galenics, antifouling), transport and separation, in sensor technology (gas sensors, moisture sensors and biosensors), substance storage (fuel cells), microelectronics (interlayer dielectrics), electronics (nanocircuits, nanocables, nanocapacitors) and in optics (light conduction, glass nanotubes for near-field optical microscopy).

According to the invention, the polymer solution is released from an applicator device, for example a spinning capillary, under pressure. For example, the polymer solution can be released manually from a syringe by means of an injection pump.

In one embodiment of the invention, the polymer solution is released by an injection pump by means of hydraulic, mechanical or pneumatic means.

In an improvement of the embodiment described above, the polymer solution can be released in an automated manner. To that end, the injection pump operated with hydraulic, mechanical or pneumatic means can be computer-controlled.

In an additional embodiment the syringe is movably arranged and can travel in the x-y-z direction.

In an improvement of the embodiment described above, the relative movement of the syringe is computer-controlled.

In an additional embodiment, the template is movably arranged and can travel in the x-y-z direction.

In a, improvement of the embodiment described above, the relative movement of the template is computer-controlled.

In an additional embodiment, both the syringe and the template are movably arranged and can travel in the x-y-z direction.

In an improvement of the embodiment described above, the relative movement of the syringe and of the template is computer-controlled.

The deposition of the nanofibers can be in a reproducible manner by means of the computer control of the relative movement of the syringe and/or of the template, which is necessary particularly in the mass production field with high quality requirements.

The invention will be described below in further detail by means of several embodiments. The attached drawings show the following:

FIG. 1 shows a schematic depiction of the conventional electrospinning process;

FIG. 2 shows a depiction of conventionally produced nanofibers;

FIG. 3 shows a depiction of a template used according to the conventional manner and of the nanofibers produced with said template;

FIG. 4 shows a depiction of a template used according to the additional conventional manner and of the nanofibers produced with said template;

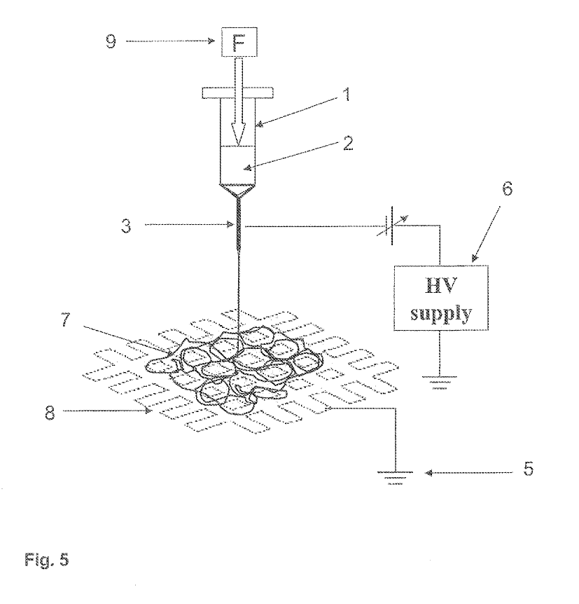

FIG. 5 shows a schematic depiction of the of spinning process according to the invention with a template;

FIG. 6 shows a schematic depiction of a template according to the invention;

FIG. 7 shows a depiction of template structures according to the invention by way of example and of the nanofiber structures according to the invention obtained with them;

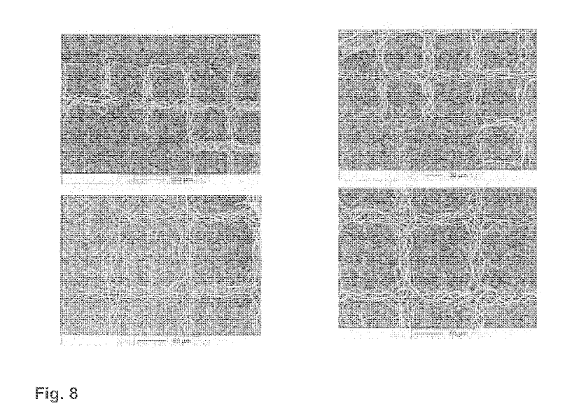

FIG. 8 shows a depiction of the nanofibers produced according to the invention.

The electrospinning device depicted in FIG. 5, which is suitable for performing the method according to the invention, comprises a syringe 1 containing a polymer molten mass 2 or solution. A spinning capillary 3 is located at the tip of the syringe 1, which is coupled with a pole of the voltage-generating arrangement 6 (current supply). The polymer molten mass or solution will transport by means of an injection pump 9 the polymer molten mass 2 or solution out of the syringe 1 towards the spinning capillary 3, where drops are accordingly formed at the tip of the spinning capillary 3. The surface tension of the drop of the polymer molten mass 2 or solution coming out of the spinning capillary 3 is overcome by means of an electric field between the spinning capillary 3 and a counter electrode 5 and then the drop coming out of the spinning capillary 3 deforms and when it reaches a critical electric potential it is drawn to yield a fine filament, the so-called jet. This electrically-charged jet, now continuously extracting new polymer molten mass 2 or solution from the spinning capillary 3 is then accelerated in the electric field towards the counter electrode 5. In this regard, it is subjected in a complex manner to bending instability (the so-called whipping mode), turned with force and highly drawn. The jet solidifies during its flight towards the counter electrode 5 by means of the evaporation of the solvent or by means of cooling, such that in the period of a few seconds continuous nanofibers 7 are generated linked with one another with typical diameters of a few nanometers to several micrometers. These nanofibers 7 are deposited on the template 8 associated with the counter electrode 5 (FIGS. 7 B, D) in the form of a web, the nonwoven mat (FIGS. 7 A, C). The conductive template 8, which is located on a standard conductive collector electrode 5, serves as a collector 4 and is grounded together with the counter electrode 5. The polymer nanofibers 7 are spun directly on the template (mold) 8. The nanofibers 7 are preferably deposited in the area of the structured template 8 in the counter electrode 5, given that the electric field intensity there has maximum values. Furthermore, the spiral-shaped line of flight of the jet upon approaching the template 8 by means of the coulometric interaction between it and the grounded template 8 or the template with the opposite charge is strictly limited to only the lattice rods in the template 8. Nanofibers are barely deposited 7 or no nanofiber is deposited, in the intermediate areas of the lattice rods in the template 8, where there is no conductive material (as in the openings of a mesh). Consequently, the deposition position can be controlled with the simultaneous patterning of the jet. If the template 8 is covered along the entire width at least once by the nanofiber 7, the spinning operation can be interrupted. Then the deposition layer of electrospun fibers 7 is carefully separated from the template 8 (FIGS. 7 B, D) to obtain the self-supporting web, the structure of which corresponds to that of the template 8 (FIGS. 7 A, C). The web which is generated in this regard is available for use or an eventual subsequent treatment. After the extraction of the web, the template 8 can be used immediately for additional electrospinning operations.

The nanofibers 7 are intertwined by means of the repetitive adjacent and overlapping placement in the form of a three-dimensional web (nonwoven mat) (FIG. 8). The size and the shape of the hollow spaces between the fibers 7 in such webs can be easily controlled by means of the choice of the template 8.

In one embodiment of the invention, the template 8 is used directly as a collector 4. The nanofibers 7 can therefore be deposited on the template 8 only in the area of the lattice rods.

In one embodiment of the invention, shown in FIG. 6, the lattice rods of the template 8, which are made, for example, as wires, wire meshes or perforated metal grids, have a ratio of width (b) of the lattice rods with respect to their thickness (d) of >1. This means that the lattice rods are wider than they are thick. The width (b) of the lattice rods characterizes in this sense the extension in direction x and/or y, whereas the thickness (d) of the lattice rods refers in this sense to the thickness of material of the lattice rods of the template 8 in direction z. In this regard it is particularly advantageous for the material of the template 8 to be essentially smaller in direction z than in direction x and/or y.

In one embodiment of the invention, active pharmaceutical ingredients are incorporated in the polymer molten masses 2 or solutions as nanoparticles before spinning with different dimensions and they are then applied, together with the polymer, on the template 8.

In an additional embodiment, the surface of the generated nanofibers 7 described above is modified by means of atomic layer deposition. Customized, application-specific nanofibers 7 can thus be generated, modifying the surface of the nanofibers 7.

In an additional embodiment of the invention, the modified nanofibers 7 described above are subjected to a thermal treatment at 500° C. in a furnace. The polymer fraction is accordingly separated from the nanofiber 7, whereby only the inorganic fraction of the nanofiber 7.

In an additional embodiment of the invention, ceramic nanofibers 7 are generated by means of the spinning process according to the invention described above. To that end, ceramic precursors of the group consisting of Al₂O₃, CuO, NiO, TiO₂, SiO₂, V₂O₅, ZnO, CO₃O₄, Nb₂O₅, MoO₃ and MgTiO₃ are added to the polymer molten mass 2 or solution and are then subjected to electrospinning. Ceramic nanofibers 7 which can be applied, for example, in composites can thus be generated.

### LIST OF REFERENCE NUMBERS

1 syringe
2 polymer molten mass or solution
3 spinning capillary
4 collector
5 counter electrode
6 current supply
7 nanodeposited fibers
8 template
9 injection pump
b width of the lattice rods of the template
d thickness of the lattice rods of the template

1. A method for producing two- and three-dimensionally structured, microporous and nanoporous webs made up of nanofibers by means of electrospinning, comprising:
providing a predefined conductive template as a collector;
generating the webs in any form with a covering or depositing degree of the nanofibers greater than 60%;
predetermining the structure of the webs to be generated by means of the template, whereby a flat template in the form of conductive lattice rods with intermediate spaces therebetween in the form of unfilled, hollow spaces is used and the template in the form of lattice rods, which are wider than they are thick, is used.

2. The method according to claim 1, wherein for obtaining the self-supporting web, the structure of which corresponds to that of the template, it is separated from the template, the template being able to be used after extracting the web immediately for additional electrospinning operations.

3. The method according to claim 1, wherein a polymer molten mass or solution is used for producing the structured webs from nanofibers, all the known natural and synthetic polymers, mixtures of polymers (polymer blends) and copolymers made up of at least two different monomers, being used as suitable polymers provided that they can be melted and/or at least be dissolved in a solvent.

4. The method according to claim 3, wherein polymers of the group consisting of polyesters, polyamides, polyimides, polyethers, polyolefins, polycarbonates, polyurethanes, natural polymers, polylactides, polyglucosides, poly-(alkyl)-methylstyrene, polymethacrylates, polyacrylonitriles, latices, poly(alkylene oxides) of ethylene oxide and/or propylene oxide and mixtures thereof are selected for producing the structured webs.

5. The method according to claim 3, wherein the polymers or copolymers are selected from the group consisting of poly-(p-xylylene); poly(vinylidene halides), polyesters such as poly(ethylene terephthalates), poly(butylene terephthalate); polyethers; polyolefins such as polyethylene, polypropylene, poly(ethylene/propylene) (EPDM); polycarbonates; polyurethanes; natural polymers, for example rubber; polycarboxylic acids; polysulfonic acids; sulfated polysaccharides; polylactides; polyglucosides; polyamides; homo- and copolymers of aromatic vinyl compounds such as poly(alkyl)styrenes, for example polystyrenes, poly-alpha-methylstyrenes; polyacrylonitriles, polymethacrylonitriles; polyacrylamides; polyimides; polyphenylenes; polysilanes; polysiloxanes; polybenzimidazoles; polybenzothiazoles; polyoxazoles; polysulfides; polyesteramides; polyarylenevinylenes; polyetherketones; polyurethanes, polysulfones, hybrid inorganic-organic polymers; silicones; fully aromatic copolyesters; poly(alkyl acrylates); poly(alkyl methacrylates); poly(hydroxyethyl methacrylates); poly(vinyl acetates), poly(vinyl butyrates); polyisoprene; synthetic rubbers such as chlorobutadiene rubbers; nitrile-butadiene rubbers; polybutadiene; polytetrafluoroethylene; modified and unmodified celluloses, homo- and copolymers of alpha-olefins and copolymers consisting of two or more of the monomer units forming the aforementioned polymers; poly(vinyl alcohols), poly(alkylene oxides), for example poly(ethylene oxides); poly-N-vinylpyrrolidone; hydroxymethylcelluloses; maleic acids; alginates; polysaccharides such as chitosans, etc.; proteins such as collagens, gelatins, their homo- or copolymers and mixtures thereof.

6. The method according to claim 3, wherein a polymer molten mass or solution of the polymers is used for producing the nanofibers, this molten mass or solution being made up of a solvent or mixtures of solvents with the polymers.

7. The method according to claim 6, wherein the solvents used are selected from the group consisting of chlorinated solvents, for example dichloromethane or chloroform; acetone; ethers, for example diethyl ether, methyl-tert-butyl ether; hydrocarbons with less than 10 carbon atoms, for example n-pentane, n-hexane, cyclohexane, heptane, octane, dimethylsulfoxide (DMSO), N-methylpyrrolidinone (NMP), dimethylformamide (DMF), formic acid, water, liquid sulfur dioxide, liquid ammonia and mixtures thereof.

8. The method according to claim 6, wherein the spinnable polymer molten masses or solutions are mixed by stirring, under the action of ultrasounds or under the action of heat.

9. The method according to claim 3, wherein the concentration of the at least one polymer in the molten mass or solution amounts to at least 0.1% by weight.

10. The method according to claim 3, wherein before spinning, nanoparticles are incorporated with different dimensions in the polymer molten masses or solutions and they are then applied, together with the polymer, on the template as nanocomposite nanofibers.

11. The method according to claim 10, wherein metals and/or semiconductors, color pigments, catalysts, active pharmaceutical ingredients, enzymes, antiviral or antibacterial active ingredients, biological messengers (such as DNA, RNA and proteins) as nanoparticles are incorporated in the polymer molten masses or solutions before spinning and they are then applied, together with the polymer, on the template.

12. The method according to claim 10, wherein ceramic nanofibers from a mixture of the polymer molten mass or solution with ceramic precursors, which are selected from the group consisting of Al₂O₃, CuO, NiO, TiO₂, SiO₂, V₂O₅, ZnO, CO₃O₄, Nb₂O₅, MoO₃ and MgTiO₃, are incorporated with different dimensions in the polymer molten masses or solutions before spinning and they are then applied, together with the polymer, on the template.

13. The method according to claim 1, wherein the webs are modified by means of chemical and/or physical processes.

14. The method according to claim 13, wherein a surface modification of the webs takes place by means of coating or irradiating with high-energy radiation, with low-temperature plasma or by means of chemical reagents, for example an aqueous hydroxide solution, inorganic acids, acyl anhydride, or halides or others depending on the surface functionality with silanes, isocyanates, organic acyl anhydrides or halides, alcohols, aldehydes or alkylating chemicals with the corresponding catalytes thereof.

15. The method according to claim 13, wherein a modification of the nanofibers in the webs takes place by enveloping the nanofibers by means of gas-phase deposition, sputtering, spin-coating, dip-coating, spraying, plasma deposition, sol-gel process or atomic layer deposition.

16. The method according to claim 1, wherein a polymer molten mass or solution is used for producing the structured webs from nanofibers, the polymer molten mass or solution being mixed with inorganic materials, then being subjected to electrospinning and the polymer fraction finally being separated from the nanofibers generated by means of electrospinning processes, whereby the remaining inorganic fractions are left as inorganic nanofibers.

17. The method according to claim 16, wherein
a two- and three-dimensionally structured, microporous and nanoporous web made up of nanofibers is generated,
a modification of the nanofibers in the webs takes place by enveloping the nanofibers by means of gas-phase deposition, sputtering, spin-coating, dip-coating, spraying, plasma deposition, sol-gel process or atomic layer deposition with an inorganic material, and
the polymer is separated after enveloping the nanofibers by means of thermal, chemical, radiation-induced, biological, photochemical processes, and also plasma, ultrasonic, hydrolysis processes or by extracting with a solvent.

18. The method according to claim 16, wherein the separation of the polymer material takes place at 10-900° C. and 0.001 mbar to 1 bar and the separation is complete or at a percentage of at least 70%.

19. A nanofiber or two- and three-dimensionally structured, microporous and nanoporous web made up of nanofibers, wherein a web is produced according to the method of claim 1.

20. The nanofiber or two- and three-dimensionally structured, microporous and nanoporous web made up of nanofibers according to claim 19, wherein the nanofiber is made up of oriented and electrospun bundles of fibers.

21. The nanofiber or two- and three-dimensionally structured, microporous and nanoporous web made up of nanofibers according to claim 20, wherein the nanofibers are joined to one another by means of adhesive forces, whereby the resulting webs together with the orientation of the fibers in the webs and the orientation of the microcrystallites, macromolecules, nanoparticles, etc. within the fibers themselves present reinforcement properties.

22. The nanofiber or two- and three-dimensionally structured, microporous and nanoporous web made up of nanofibers according to claim 21, wherein the nanofibers present a covering or depositing degree of the nanofibers in the range between 60 and 100%.

23. The nanofiber or two- and three-dimensionally structured, microporous and nanoporous web made up of nanofibers according to claim 19, wherein the nanofibers are made up of at least one polymer selected from the group consisting of polyesters, polyamides, polyimides, polyethers, polyolefins, polycarbonates, polyurethanes, natural polymers, polysaccharides, polylactides, polyglucosides, poly-(alkyl)-methylstyrene, polymethacrylates, polyacrylonitriles, latices, poly(alkylene oxides) of ethylene oxide and/or propylene oxide and mixtures thereof.

24. The nanofiber or two and three-dimensionally structured, microporous and nanoporous web made up of nanofibers according to claim 19, wherein the nanofibers are enveloped by means of gas-phase deposition, sputtering, spin-coating, dip-coating, spraying, plasma deposition, sol-gel process or atomic layer deposition.

25. The nanofiber or two- and three-dimensionally structured, microporous and nanoporous web made up of nanofibers according to claim 19, wherein the nanofibers have at least one inorganic component.

26. The nanofiber or two- and three-dimensionally structured, microporous and nanoporous web made up of nanofibers according to claim 19, wherein the nanofibers have functionalizations with nanoparticles in the form of pigments, dyes, chromophores, catalysts, messengers, inorganic materials, metals, conductive materials, ceramic precursors, magnetic particles, semiconductor materials, pharmaceutically active ingredients, fragrant substances, messengers, proteins, enzymes, DNA, RNA, mRNA, substances with antibiotic action, biocompatible materials or mixtures thereof.

27. The nanofiber or of the two- and three-dimensionally structured, microporous and nanoporous web made up of nanofibers according to claim 19 used in the following applications: filters or parts of filters; electrical and optoelectrical applications; in microelectronics, electronics, photovoltaics, optics; photovoltaic applications; semiconducting polymers for polymer electronics, in field-effect transistors, computer chips, display technology, electromagnetic interference shielding, in communication networks, for use in high-density data storage media, magnetic logic junctions, spintronic devices; magnetic sensors and magnetic composites; in sensor technology; as a textile material coating or component for technical, medical or domestic textile materials; component of composites; as a component of ultra-lightweight nanocomposites; in biotechnological applications; corrosion protection; as a semiconductor; in the medical and pharmaceutical field, active ingredient transport and release, as support tubes for regenerating blood vessels, the esophagus and nerves, support tubes with pharmaceutically active substances, for implant surface modification; transport and separation, for use in wound healing or as a dressing for wounds, as wound-specific plaster with special active ingredients for the treatment of chronic diseases, as porous membranes and temporary skin graft, in medical diagnostic applications, in the targeted application of magnetic field-controlled active ingredients, in hyperthermic treatment, as magnetically switchable bioelectrocatalytic systems; as catalyst supports for catalytic processes; substance storage; fuel cells, ceramic materials.

28. A device for performing a method according to claim 1, wherein, with an electrospinning device with a spinning capillary and a collector, which is configured as a counter electrode with respect to the spinning capillary, and with a voltage-generating arrangement that generates an electrical voltage between the spinning capillary and the collector, whereby a predefined, structured conductive template, corresponding to the structure of the nanofibers to be generated, is detachably arranged as a collector on the conductive counter electrode or forms the counter electrode, characterized in that the template is configured flat and in the form of conductive lattice rods with intermediate spaces therebetween in the form of unfilled, hollow spaces, and in that the lattice rods of the template are wider than they are thick.

29. The device for performing a method according to claim 28, wherein the template is made up of a conductive material which is in the form, for example, of wires and wire meshes or perforated metal grids, etc., of semiconductors or metal materials or in the form of fabrics made up of natural or chemical fibers, impregnated with a conductive agent to increase conductivity thereof.

30. The device for performing a method according to claim 28, wherein the template is produced by means of conventional micromanufacturing techniques.
